(19) United States
(12) Patent Application Publication
(10) Pub. No.: US 2025/0388982 A1
(43) Pub. Date: Dec. 25, 2025

(54) Method and Apparatus for Detection of Microorganisms Using Metagenomics

(71) Applicants: Guy's and St Thomas’ NHS Foundation Trust, London (GB); Synnovis Group LLP, London (GB)

(72) Inventors: Rahul BATRA, London (GB); Adela ALCOLEA-MEDINA, London (GB)

(73) Assignees: Guy's and St Thomas’ NHS Foundation Trust, London (GB); Synnovis Group LLP, London (GB)

(21) Appl. No.: 18/869,626

(22) PCT Filed: May 30, 2023
(86) PCT No.: PCT/GB2023/051417

§ 371 Date: Nov. 26, 2024

(30) Foreign Application Priority Data
May 30, 2022 (GB) 2207989.1

(51) Int. Cl. C12Q 1/70; C12Q 1/689; C12Q 1/6895

(52) U.S. Cl. CPC C12Q 1/701; C12Q 1/689; C12Q 1/6895

(57) ABSTRACT

The present invention relates to metagenomics methods. The invention also relates to their use in the detection and/or diagnosis of a wide range of infectious diseases, for example, caused by a pathogenic virus, bacterium, fungus or protozoan. The invention also extends to methods for sample preparation and microorganism detection and/or identification, and methods for host cell nucleic acid depletion. The invention further relates to kits and apparatus used in these methods. The invention is especially useful in clinical diagnostic and veterinary medicine.

This application is a 35 U.S.C. 371 National Phase Entry Application from PCT/GB2023/051417 filed May 30, 2023, which claims priority to and the benefit of GB Patent Application No. 2207989.1 filed on May 30, 2022, the disclosures of which are incorporated herein by reference in their entirety.

The instant application contains a Sequence Listing which is hereby incorporated by reference in its entirety. Said Sequence Listing, created on Jun. 7, 2023, is named 124081PCT1.XML and is 12317 bytes in size.

The present invention relates to metagenomics methods and particularly, although not exclusively, to their use in the detection and/or diagnosis of a wide range of infectious diseases, for example, caused by a pathogenic virus, bacterium, fungus or protozoan. The invention also extends to methods for sample preparation and microorganism detection and/or identification, and methods for host cell nucleic acid depletion. The invention further relates to kits and apparatus used in these methods. The invention is especially useful in clinical diagnostic and veterinary medicine.

Infectious diseases are still one of the most common causes of morbidity and mortality in humans and animals. Many classical infections, such as cholera and typhoid, persist in developing countries, and new diseases such as Ebola are emerging. In developed countries, infections associated with zoonosis or healthcare systems are an ever-present threat, and given the increasing age demographic, these threats are unlikely to recede [5, 6]. Furthermore, the global emergence of multiple drug-resistant microbes is challenging our ability to diagnose and treat infections safely.

Moreover, the emergence of novel and rare pathogens and associated diseases, such as SARS-COV-2, monkeypox virus (MPXV), or adenovirus-related severe hepatitis, highlights the need for having methods for rapid, untargeted identification and characterisation of pathogens [SARS NEJM] in order to improve patient management, implement a targeted therapy in the least amount of time, and prevents disease spreading in the hospital ward, the whole hospital or beyond. Additionally, the ability to rapidly identify life-threatening pathogens in clinical settings from a variety of sample matrixes, such as blood, sputum and various swabs is of paramount importance.

Currently, the “gold standard” method for pathogen identification is microbial culture. This method, however, presents major limitations, such as poor sensitivity, significantly time-consuming, labour intensive and higher costs of implementation. Alongside microbial culture, molecular diagnostic methods based on nucleic acid amplification tests (NAATs), such as the Polymerase Chain Reaction (PCR), have been developed and are successfully used in clinical diagnosis. Although these methods present a significant improvement in the time and sensitivity of microbial cultures, they still have a limited range of application, and hence, rare pathogens and resistance markers are rarely identified with such methods. These technologies include Septifast (RTM, Roche), used to detect sepsis. However, its complexity of use and suboptimal performance have prevented its widespread adoption. NAATs targeting respiratory tract infections have also been developed; examples of these technologies include Biofire Filmarray Respiratory Panel, Seegene RV15, targeting respiratory viruses, and Curetis Unyvero, designed to identify pneumonia-causing bacteria. However, these methods can only detect a limited range of target pathogens. Therefore, rare pathogens are unlikely to be captured by these technologies.

More recently, metagenomic methods have been developed and are increasingly used to analyse complex metagenomes in clinical samples. Metagenomic techniques enable detecting and/or identifying different pathogens such as viruses, bacteria, fungi and protozoans directly from a sample without any prior knowledge of the microbial community present in the sample. However, these methods also present some significant disadvantages. Notably, the time required to perform a diagnosis from sample collection to detection of the pathogen nucleic acid can often average one week. This is mainly due to the high quantity of human DNA present in the clinical sample, sequenced simultaneously with microbial DNA and RNA.

To address these limitations, several sample preparation methods have been developed, including human DNA depletion or preliminary nucleic acid extraction and sequencing. However, these methods only enable the detection of microbial DNA or RNA, and not both types of nucleic acid simultaneously. This is because, in these techniques, the human DNA depletion step employs different chemicals applied to lyse the human cells, which also affects the microorganisms present in the sample. In addition, some of these methods also include centrifugation steps, which splits the microorganism content of the sample into two phases; bacteria are likely to sediment and form a deposit in the testing tube, whereas viruses are likely to be present in the supernatant. Therefore, collecting the total microorganism load in the sample becomes difficult.

There is, therefore, a need to address the problems in the art, and to provide improved methods for metagenomics analysis of samples, and sample preparation.

Accordingly, in a first aspect of the invention, there is provided a method for detecting a microorganism in a biological sample obtained from a mammalian host, the sample comprising mammalian host cells and a microorganism, the method comprising:
(i) subjecting the biological sample to mechanical disruption such that the mammalian host cells are lysed to thereby release host's nucleic acid while leaving the microorganism substantially intact;
(ii) contacting the mechanically disrupted sample with a nuclease, to thereby digest the host's released nucleic acid while leaving the microorganism's nucleic acid substantially intact;
(iii) extracting the micro-organism's nucleic acid from the sample; and
(iv) detecting the extracted micro-organism's nucleic acid.

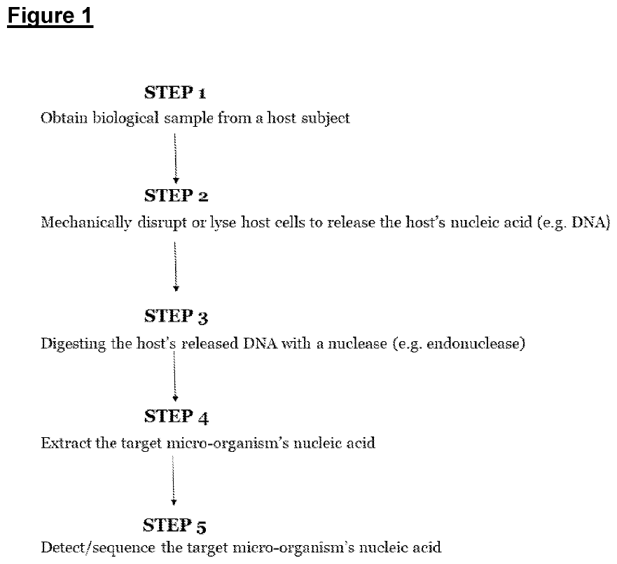

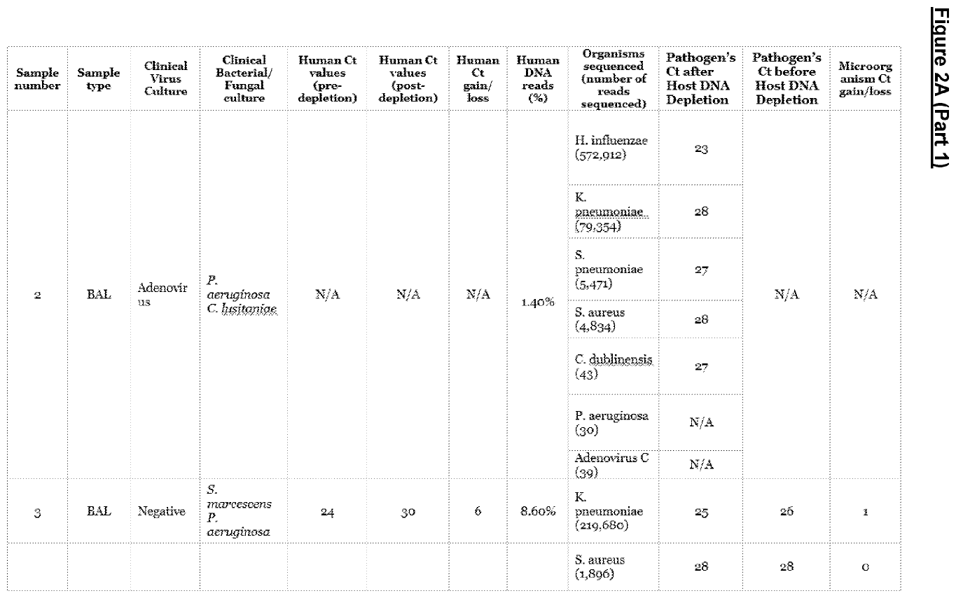

Advantageously, in order to overcome the limitations of existing metagenomic methods, the inventors have developed the method of the invention (an embodiment of which is shown in FIG. 1) in which mechanical disruption is used in an initial step of depleting or removing the host's (e.g. human) nucleic acid (in particular the genomic DNA), thus allowing the subsequent detection and/or identification of the infecting microorganism that is also present in a clinical sample. The method is non-specific in that it does not involve specifically targeting a certain pathogenic microorganism in the sample. However, surprisingly, the method makes it possible, for the first time, to not only distinguish different infecting pathogenic microorganism (e.g. any target bacteria, fungi, viruses and protozoans) but also different types of microbial nucleic acid, (i.e. RNA and/or DNA), which are simultaneously present in the same sample, and rapidly achieve sufficient genome coverage for genomic epidemiology studies. The method results in microbial detection and/or identification in record time (i.e. only 7 hours) directly from a clinical sample. The method also enables the detection of infectious pathogens in a broad range of biological samples with no prior knowledge of the microbial community that is present in the sample. Furthermore, advantageously, in some embodiments, no centrifugation is required in the method unlike in prior art methods.

Known methods relying on the chemical lysis of the host cells to release their DNA often require the use of lysing agents or detergents, which can be unsuitable in clinical settings. Advantageously, the inventors have discovered that the use of mechanical disruption in the method of the invention decreases the risk of contamination that is otherwise associated with prior art methods relying on chemical lysis of host cells to release their genomic nucleic acids (preferably DNA), where adequate concentrations of the lysing agent are prepared prior to the sample treatment. Also, the use of mechanical disruption allows the detection of different types of nucleic acid (i.e. DNA/RNA) simultaneously, with only minimal manipulation of the sample being required.

In a second aspect of the invention, there is provided a method for depleting host nucleic acid in a biological sample obtained from a mammalian host, the sample comprising mammalian host cells and a microorganism, the method comprising:
(i) subjecting the biological sample to mechanical disruption such that the mammalian host cells are lysed to thereby release host's nucleic acid while leaving the microorganism substantially intact; and
(ii) contacting the mechanically disrupted sample with a nuclease, to thereby digest and deplete the host's released nucleic acid while leaving the microorganism's nucleic acid substantially intact.

In the method of the first or second aspect, it is the host's RNA, which is depleted by the nuclease digestion step. However, preferably it is the host's genomic nucleic acid (i.e. DNA) which is depleted by the nuclease digestion step.

The sample may be any biological material that is obtainable from a mammalian host. For example, the sample may be blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, or combinations thereof. The sample may comprise a swab from any part of the body, such as skin swabs or rectal swabs, as well as tissues or cells isolated from any part of the body and any biological fluid.

Preferably, however, the sample is a respiratory sample, such as pleural fluid (PF), Bronchoalveolar lavage (BAL), sputum, non-direct Bronchoalveolar lavage (NDL), or nose, mouth or throat swabs.

The mammalian host may be a vertebrate, or domestic animal. The invention may be used to detect pathogenic microbial infections in any mammal, for example livestock (e.g. a horse, or pig), pets, or may be used in other veterinary applications. Most preferably, however, the host is a human being. The host may be male or female.

The mechanical disruption step (i) of the method may be performed by various means in order to preferentially release the host's nucleic acid from the host cells but leaving the pathogenic microorganism's nucleic acid preserved for subsequent detection or sequencing. Preferably, the mechanical disruption step is achieved by contacting the sample with a plurality of particles, and then agitating the resultant sample for sufficient time and at sufficient intensity so that the particles cause the mammalian host cells to lyse, thereby releasing their nucleic acid (preferably genomic DNA). Preferably, the particles selectively disrupt substantially only the host cells, while preserving the infecting microorganism intact, thereby protecting their DNA/RNA.

Preferably, at least 100, 500 or 1000 particles are used. More preferably, at least 2000,3000 or 5000 particles are used.

The particles may comprise stainless steel, ceramic or glass. Preferably, the particles comprise ceramic. Preferably, the particles comprise microspheres or beads. The average diameter of the particles may be between about 1 mm and 2 mm, more preferably between about 1.1 mm and 1.8 mm, even more preferably between about 1.2 mm and 1.6 mm, and most preferably between about 1.3 mm and 1.5 mm. Most preferably, the average particle diameter is about 1.4 mm.

Preferably, the average particle capacity is between 0.5 and 5 ml, or between 1 and 4ml, or between 1.5 and 3 ml. Preferably, the average particle capacity is about 2 ml. Preferably, the average particle hardness is Vickers Hardness is 800.

In some embodiments, the methods of the first or second aspect may comprise initially centrifuging the biological sample before it is subjected to the mechanical disruption.

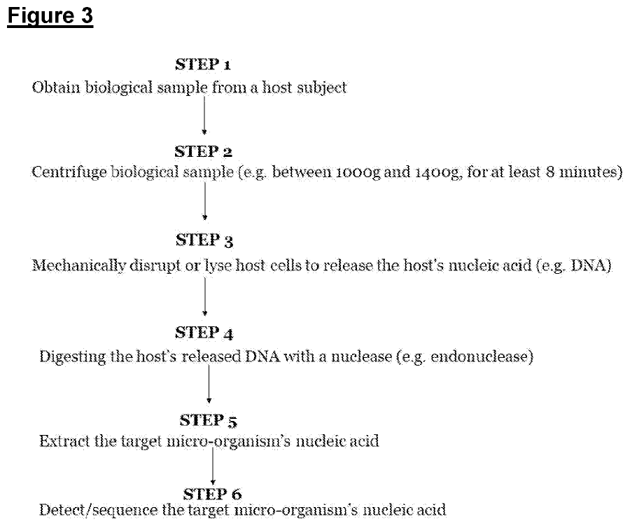

An embodiment of the method comprising such a centrifugation step is shown in FIG. 3).

Thus, the method may comprise:
(i) centrifuging the biological sample;
(ii) subjecting the biological sample, preferably a supernatant thereof, to mechanical disruption such that the mammalian host cells are lysed to thereby release host's nucleic acid while leaving the microorganism substantially intact;
(iii) contacting the mechanically disrupted sample with a nuclease, to thereby digest the host's released nucleic acid while leaving the microorganism's nucleic acid substantially intact;
(iv) extracting the micro-organism's nucleic acid from the sample; and
(v) detecting the extracted micro-organism's nucleic acid.

Advantageously, the initial centrifugation step makes the subsequent mechanical disruption step more efficient and removes human cells from the supernatant. Preferably, the initial centrifugation step is conducted at a relative centrifugal force or G force (g) of between 300 g and 2000 g, more preferably between 500 g and 1800 g, even more preferably between 700 g and 1600 g, and most preferably between 1000 g and 1400 g. Most preferably, however, the preliminary centrifugation of the sample is conducted at about 1200 g.

Preferably, the preliminary centrifugation step is carried out for at least 1 minute, at least 2 minutes, or at least 5 minutes. More preferably, the preliminary centrifugation step is carried out for at least 7 minutes, at least 9 minutes, or at least 10 minutes.

It will be appreciated that any of the above durations of centrifugation can be combined with any of the above centrifugal forces. For example, preferably the initial centrifugation step is conducted at between 300 g and 2000 g for at least 1 minute, or between 500 g and 1800 g for at least 5 minutes, or preferably between 1000 g and 1400 g for at least 8 minutes. Most preferably, the initial centrifugation is at about 1200 g for about 10 minutes. This is referred to as a “slow centrifugation”.

In contrast, methods disclosed in the prior art require a centrifugation of the sample at high speed in order to concentrate the bacteria and fungi in the deposit to increase the sensitivity of the detection of those microorganisms. In these methods, the deposit or pellet is used to detect the microorganism instead of the supernatant. Therefore, these methods cannot detect viruses given that these smaller organisms are contained in the supernatant after the centrifugation.

Preferably, the method of the first or second aspect comprises agitating the biological sample (or the supernatant resulting from the preliminary centrifugation of the sample) in the presence of the plurality of particles in a container. Preferably, the volume of the biological sample (or the supernatant resulting from the preliminary centrifugation of the sample) is at least 100 μl, 200 μl or 300 μl. Preferably, the volume of the biological sample is at least 400 μl or 500 μl. The volume of the biological sample may preferably be at least 1000 μl, 1500 μl or 2000 μl.

As described in the examples, the preferred particles used in the agitation step are comprised in MP biomatrix Lysis matrix tubes D, sourced from MP Biomedicals™.

Lysis matrix tubes D are ideal for sample bead-beating using the TissueLyser LT, which provides fast, effective mechanical disruption for up to 12 samples concurrently.

Preferably, the sample may be agitated at at least 5, 10, 15 or 20 oscillations per second (OSC/sec) for sufficient time as described below. Preferably, the sample may be agitated at at least 25, 30 or 35 oscillations per second (OSC/sec) for sufficient time. Preferably, the sample may be agitated at at least 40, 45 or 50 oscillations per second (OSC/sec). Preferably, the sample may be agitated at less than 200, 150, 100 or 75 oscillations per second (OSC/sec). Preferably, the sample may be agitated for at least 15, 30, 45 or 60 seconds at the above-mentioned speeds. Preferably, the sample may be agitated for at least 1 min and 15 s, 1 min and 30 s, 1 min and 458, or 2 mins at the above-mentioned speeds. Preferably, the sample may be agitated for at least 2 min and 15 s, 2 min and 30 s, 2 min and 45 s, or 3 mins at the above-mentioned speeds. Preferably, the sample may be agitated for less than 30 min, 20 min, 10 min or 5 min at the above-mentioned speeds.

The mechanical disruption step is preferably carried out at room temperature. Preferably, the temperature of the mechanical disruption step is between 12° C. and 25° C., preferably about 20° C. or 21° C.

Preferably, step (ii) comprises contacting the disrupted sample with a nuclease to digest the host's nucleic acid (preferably genomic nucleic acid, i.e. DNA) that has been released by the mechanical agitation step. Preferably, the nuclease is added and allowed to act for a period of time such that sufficient host nucleic acid digestion can occur. Preferably, therefore, a deoxyribonuclease (DNase) and/or a ribonuclease (RNase) is contacted with the sample (and preferably allowed to act for a period of time such that sufficient DNA/RNA digestion can occur). The nuclease may therefore have both DNase and RNase activity (e.g. HL-SAN DNase). Depletion of host nucleic acid DNA is important if analysis of the infecting pathogen (i.e. non-host or pathogen) DNA or RNA is to be carried out. Depletion of host RNA is important if analysis of pathogen (i.e. non-host or pathogen) RNA is to be carried out, and indeed can facilitate the optimisation of DNA analysis (e.g. DNA sequencing).

The nuclease may be an endonuclease or an exonuclease (or a combination thereof can be provided), but is preferably an endonuclease. Preferred DNases (particularly where the biological sample is a blood sample) may comprise HL-SAN DNase (heat-labile salt activated nuclease, supplied by Arcticzymes) and MolDNase (endonuclease active in the presence of chaotropic agents and/or surfactants, supplied by Molzym), and active variants are also contemplated.

Preferably, the sample is subjected to mixing after the nuclease has been added. Mixing may be achieved, for example, by spinning the sample and nuclease at at least 100 rpm, preferably at least 500 rpm, and more preferably at least 1000 rpm.

Preferably, to promote nuclease activity, particular buffering conditions and/or incubation temperature might be provided for any one selected nuclease. Nuclease incubation can take place at e.g. between 5° C. and 50° C., such as between 15° C. and 45° C., preferably between 30 and 40° C. (e.g. 37° C.), and for between 1 min and 120 min, preferably between 2 min and 60 min, more preferably between 3 min and 30 min, and even more preferably between 5 min and 20 min (e.g. 10 min). In particularly preferred embodiments, a nuclease buffer is added to the sample and incubated (e.g. as described above).

Preferably, the nuclease comprises a non-specific nuclease, i.e. non-specific DNase and/or non-specific RNase. This means that the enzyme digests the host's nucleic acid in a non-specific manner, i.e. it digests the DNA present in the sample due to its DNase activity and any RNA present due to its RNase activity. The infecting microorganisms are intact because the mechanical disruption step (i) does not affect them, and so their DNA or RNA are not digested by the nuclease in step (ii).

Although high salt concentrations present significant advantages in protein purification, they can negatively affect the microorganisms present in the sample because of its dehydrating effects (and therefore killing) on the microorganism cells. The inventors have surprisingly discovered that using little to no salt during the enzymatic digestion of the host nucleic acids better preserves the pathogen community that is also present in the sample. Additionally, the absence of salt during the enzymatic digestion step ensures that there is minimal impact on the structure and function of the pathogens' nucleic acid allowing better subsequent detection and diagnosis.

In one preferred embodiment, therefore, the nuclease is used with or without a salt, such as sodium chloride (NaCl). Preferably, the concentration of salt is less than 2 M, or less than 1 M. Preferably, the concentration of salt is less than 0.75 M, or less than 0.5 M salt. More preferably, the concentration of salt is less than 0.25 M, or less than 0.1 M salt. Preferably, however, the nuclease is used without a salt. One example of non-specific endonuclease HL-SAN, a non-specific endonuclease active in various buffers and can be easily inactivated by treatment with a reducing agent. These features make HL-SAN particularly useful in protein purification and removal of both DNA and RNA from molecular biology reagents. Accordingly, in a preferred embodiment, the non- specific nuclease is HL-SAN. Nucleic acids, especially genomic DNA, often pose a problem in the purification of DNA-binding proteins as they interfere with purification, downstream analysis or applications. In addition, contrary to most endonucleases, HL-SAN exhibits an optimum activity at high salt concentrations.

The high salt-tolerance and easy removal make HL-SAN beneficial to use in protein purification schemes, microorganism bioprocessing, PCR carry-over prevention, isothermal amplification, contamination control in RT-lamp, removal of genomic DNA from RNA preparations, decontamination of PCR master mix, PCR product clean-up, dephosphorylation before cloning, complete removal of DNA and RNA, and viscosity reduction.

Therefore, in a preferred embodiment, HL-SAN is used with little to no salt.

The method may further comprise a subsequent step of neutralising the nuclease (i.e. decreasing or substantially eliminating the activity of the nuclease). The skilled person will appreciate a range of neutralisation options, to be selected for each depletion protocol. This might include heat inactivation or, preferably, buffer exchange (i.e. the removal of a buffer in which the nuclease is active and/or replacement with or addition of a buffer in which the nuclease is substantially inactive). Preferably, the temperature of the sample (at any/all stage(s) at/before extraction of remaining nucleic acid from the sample) is maintained at 50° C. or less, preferably 45° C. or less, preferably 40° C. or less, to optimise subsequent release of nucleic acid from the pathogen (particularly from bacterial cells).

The micro-organism's nucleic acid, which may be DNA or RNA, may be extracted in step (iii) from the digested sample using any automatic instrument or any manual extraction kit. As described in the examples, the inventors used MAgnapure from Roche, which it is an automatic instrument which extracts DNA and/or RNA from any type of clinical sample.

The method is non-specific in that it does not involve specifically targeting a certain pathogenic microorganism in the sample. Thus, the detection step (iv) is non-specific. Preferably, however, the method comprises a step of sequencing the pathogenic microorganism's nucleic acid, thereby detecting the microorganism.

In an embodiment in which the microorganism's nucleic acid is sequenced, the method may comprise initially converting its DNA or its RNA to complementary DNA (cDNA). For RNA, a subsequent second strand synthesis of the cDNA is required, which is then sequenced. Thus, in embodiments where the microorganism is RNA, it is preferably converted into double stranded DNA before library preparation occurs.

The method preferably comprises sequencing the microorganism's nucleic acid using Oxford Nanopore Technology (ONT), Rapid sequencing DNA-PCR barcoding kit SQK-RP004. Firstly, the dsDNA present is the sample is preferably fragmented, followed by a PCR reaction, However, the PCR reaction has been adapted for this method, as the extension time for the PCR is 4 min instead of 6 min and the number of PCR cycles has been increased up to 30 from the 14 cycles recommended by ONT.

Preferably, therefore, the method comprises a PCR reaction which takes fewer than 6 mins, preferably fewer than 5 mins. Preferably, the method comprises subjecting the DNA to at least 15 cycles, preferably at least 18 cycles, more preferably at least 20 cycles. Preferably, the method comprises subjecting the DNA to at least 23 cycles, preferably at least 25 cycles, more preferably at least 27 cycles, and most preferably at least 30 cycles.

The PCR reaction comprises the use of primers which target any dsDNA present in the extracted sample. Rapid adapters are preferably added to the PCR products and which are then sequenced according to the manufacturer's instructions. The kit is provided with 12 different barcodes, which act as the primers:

Barcodes:

| Component | Sequence |
|---|---|
| BC01 | AAGAAAGTTGTCGGTGTCTTTGTG [SEQ ID No: 1] |
| BC02 | TCGATTCCGTTTGTAGTCGTCTGT [SEQ ID No: 2] |
| BC03 | GAGTCTTGTGTCCCAGTTACCAGG [SEQ ID No: 3] |
| BC04 | TTCGGATTCTATCGTGTTTCCCTA [SEQ ID No: 4] |
| BC05 | CTTGTCCAGGGTTTGTGTAACCTT [SEQ ID No: 5] |
| BC06 | TTCTCGCAAAGGCAGAAAGTAGTC [SEQ ID No: 6] |
| BC07 | GTGTTACCGTGGGAATGAATCCTT [SEQ ID No: 7] |
| BC08 | TTCAGGGAACAAACCAAGTTACGT [SEQ ID No: 8] |
| BC09 | AACTAGGCACAGCGAGTCTTGGTT [SEQ ID No: 9] |
| BC10 | AAGCGTTGAAACCTTTGTCCTCTC [SEQ ID No: 10] |
| BC11 | GTTTCATCTATCGGAGGGAATGGA [SEQ ID No: 11] |
| BC12 | CAGGTAGAAAGAAGCAGAATCGGA [SEQ ID No: 12] |
| RLB12A | GTTGAGTTACAAAGCACCGATCAG [SEQ ID No: 13] |

In one embodiment, the microorganism may comprise a plurality of microorganisms in the sample. Preferably, the microorganism or plurality of microorganisms may be selected from a bacterium, virus, fungus and/or protozoan. In some embodiments, the sample may comprise one type of pathogen, e.g. a bacteria, viruses, fungi and/or protozoa, but in other embodiments, the sample may comprise two or more types of pathogen, e.g. a bacteria, viruses, fungi and/or protozoa.

The bacterium may be a Gram positive or a Gram negative bacterium. The bacterium may be a mycobacterium or a bacterium without a cell wall.

The bacterium may be selected from the group consisting of: Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus inkuenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediaeJFrancisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enter ocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorfer, Porphyromonas s and Klebsiella sp. Preferably, the bacterium is selected from the group consisting of Streptococcus pneumoniae, Mycobaterium tuberculosis or Heamophilus Influenzae.

The virus may be a DNA virus or an RNA virus. The virus may be selected from the group consisting of Orthomyxoviruses; Paramyxoviridae viruses; Metapneumovirus and Morbilliviruses; Pneumoviruses; Paramyxoviruses; Poxviridae; Metapneumoviruses; Morbilliviruses; Picornaviruses; Enteroviruseses; Bunyaviruses; Phlebovirus; Nairovirus; Heparnaviruses; Togaviruses; Alphavirus; Arterivirus; Flaviviruses; Pestiviruses; Hepadnaviruses; Rhabdoviruses; Caliciviridae; Coronaviruses; Retroviruses; Reoviruses; Parvoviruses; Delta hepatitis virus (HDV); Hepatitis E virus (HEV); Human Herpesviruses and Papovaviruses.

The Orthomyxoviruses may be Influenza A, B and C. The Paramyxoviridae virus may be Pneumoviruses (RSV), Paramyxoviruses (PIV). The Metapneumovirus may be Morbilliviruses (e.g., measles). The Pneumovirus may be Respiratory syncytial virus (RSV), Bovine respiratory syncytial virus, Pneumonia virus of mice, or Turkey rhinotracheitis virus. The Paramyxovirus may be Parainfluenza virus types 1-4 (PIV), Mumps, Sendai viruses, Simian virus 5, Bovine parainfluenza virus, Nipahvirus, Henipavirus or Newcastle disease virus. The Poxviridae may be Variola vera, for example Variola major and Variola minor. The Metapneumovirus may be human metapneumovirus (hMPV) or avian metapneumoviruses (aMPV). The Morbillivirus may be measles. The Picornaviruses may be Enteroviruses, Rhinoviruses, Heparnavirus, Parechovirus, Cardioviruses and Aphthoviruses. The Enteroviruses may be Poliovirus types 1, 2 or 3, Coxsackie A virus types 1 to 22 and 24, Coxsackie B virus types 1 to 6 , Echovirus (ECHO) virus) types 1 to 9, 11 to 27 and 29 to 34 or Enterovirus 68 to 71. The Bunyavirus may be California encephalitis virus. The Phlebovirus may be Rift Valley Fever virus. The Nairovirus may be Crimean-Congo hemorrhagic fever virus. The Heparnaviruses may be Hepatitis A virus (HAV). The Togaviruses may be Rubivirus. The Flavivirus may be Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus or Powassan encephalitis virus. The Pestivirus may be Bovine viral diarrhea (BVDV), Classical swine fever (CSFV) or Border disease (BDV). The Hepadnavirus may be Hepatitis B virus or Hepatitis C virus. The Rhabdovirus may be Lyssavirus (Rabies virus) or Vesiculovirus (VSV). The Caliciviridae may be Norwalk virus, or Norwalk-like Viruses, such as Hawaii Virus and Snow Mountain Virus. The Coronavirus may be SARS COV-1, SARS-COV-2, MERS, Human respiratory coronavirus, Avian infectious bronchitis (IBV), Mouse hepatitis virus (MHV), or Porcine transmissible gastroenteritis virus (TGEV). The Retrovirus may be Oncovirus, a Lentivirus or a Spumavirus. The Reovirus may be an Orthoreo virus, a Rotavirus, an Orbivirus, or a Coltivirus. The Parvovirus may be Parvovirus B 19. The Human Herpesvirus may be Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), or Human Herpesvirus 8 (HHV8). The Papovavirus may be Papilloma viruses, Polyomaviruses, Adenoviruses or Arenaviruses. Preferably, the virus is selected from the group consisting of SARS COV, SARS COV2, MERS or Influenza.

The fungus may be selected from the group consisting of Dermatophytres, including: Epidermophyton koccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme; or from Aspergillus fumigatus, Aspergillus kavus, Aspergillus niger, Aspergillus nidulans, Aspergillus terreus, Aspergillus sydowi, Aspergillus kavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malasseziaspp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp. Preferably, the fungi is selected from the group consisting of Aspergillus, Cryptococcus, or Pneumocystis.

The protozoan may be selected from the group consisting of: Entamoeba histolytica, Giardia lambli, Cryptosporidium parvum, Cyclospora cayatanensis and Toxoplasma.

In a third aspect of the invention, there is provided an apparatus for detecting a microorganism in a biological sample comprising mammalian host cells and a microorganism, the apparatus comprising:
a mechanical cell disruptor configured, in use, to mechanically disrupt a biological sample such that the mammalian host cells are lysed to thereby release host's nucleic acid while leaving the microorganism substantially intact; and
a nuclease configured, in use, to thereby digest the host's released nucleic acid while leaving the microorganism's nucleic acid substantially intact.

The apparatus preferably comprises reagents for detecting (and optionally sequencing) the microorganism's nucleic acid.

Preferably, mechanical cell disruptor comprises a plurality of particles and a device for mechanically agitating the particles in the sample.

The apparatus is preferably configured to carry out the method according to either the first or second aspect.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figure, in which:

FIG. 1 provides a schematic overview of one embodiment of a method according to the invention for detecting a micro-organism in a sample taken from an infected host subject, such as a human patient. The infecting micro-organism can be a bacterium, fungus and/or DNA or RNA virus.

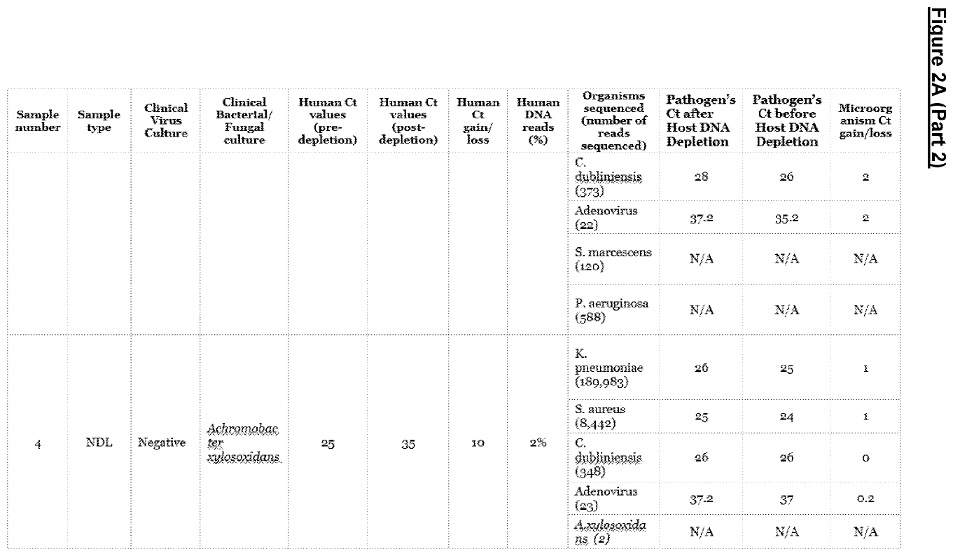

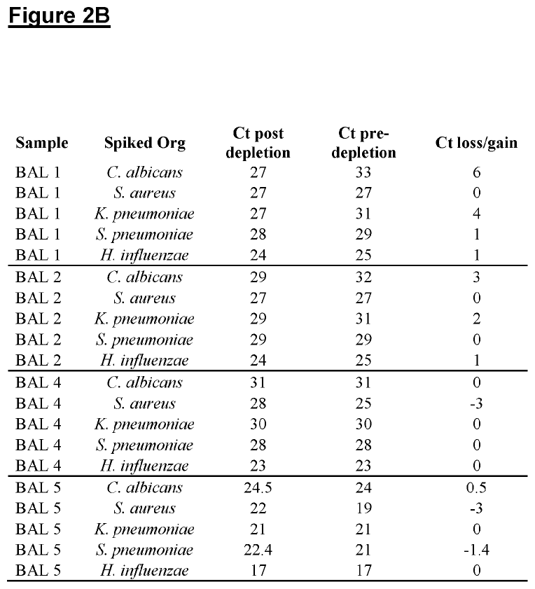

FIG. 2A shows the simultaneous detection of both DNA and RNA in clinically positive respiratory samples, further spiked with different pathogens. The Ct (cycle threshold) is defined as the number of cycles required for the fluorescent signal to cross the threshold (i.e. exceeds background level). Ct levels are inversely proportional to the amount of target nucleic acid present in the sample, which means that the lower the Ct level, the greater the quantity of target nucleic acid present in the sample. The Ct loss values are proportional to the amount of target nucleic acids lost during the process (i.e. the lower the Ct loss value, the lower the loss of target nucleic acids; conversely, higher Ct loss values correspond to higher loss or depletion of the target nucleic acid).

FIG. 2B shows spiked organisms into clinically negative BAL samples.

FIG. 3 provides a schematic overview of another embodiment of a method according to the invention for detecting a micro-organism in a sample taken from an infected host subject, such as a human patient. The infecting micro-organism can be a bacterium, fungus and/or DNA or RNA virus.

### EXAMPLES

The inventors have produced a novel method and kit in which bacteria, fungi and both DNA & RNA viruses (i.e. a target micro-organism) can be detected in the same sample using a mechanical cell disruption step paired with human host DNA and RNA removal without the use of centrifugation.

Referring to FIG. 1, there is shown one embodiment of the method of the invention in which:
1) Step 1 involves taking a biological sample from a host subject;
2) Step 2 involves mechanically disrupting or lysing the host cells to release the host's nucleic acid (including the genomic DNA);

3) Step 3 involves digesting the host's released nucleic acid with an nuclease;
4) Step 4 involves extraction of the infecting micro-organism's nucleic acid; and
5) Step 5 involves detecting and/or sequencing the micro-organism's nucleic acid.

Optionally, referring to FIG. 3, for some samples, step 2 can be preceded by a preliminary slow centrifugation of the biological sample prior to mechanically disrupting or lysing the host cells remaining in the supernatant resulting from the preliminary slow centrifugation in order to release the host's nucleic acid. Materials and Methods

### Step 1—Sample Preparation

To demonstrate that the methods and kits of the invention could efficiently and rapidly discriminate nucleic acids of the infecting microorganisms from those of the host in clinical settings, the inventors tested the depletion of human DNA and RNA in various biological samples, including respiratory samples consisting of pleural fluids (PF), Bronchoalveolar lavage (BAL), sputum, non-direct Bronchoalveolar lavage (NDL), nose, mouth and throat swabs. Different sample types can also be considered, including swabs from any part of the body, such as skin swabs or rectal swabs, as well as tissues or cells isolated from any part of the body and any biological fluid.

As shown in FIG. 3, an initial slow centrifugation step (step 2) at 1200 g for 10 minutes may be performed on the sample in order to make the subsequent mechanical disruption method more efficient and remove human cells from the supernatant.

### Step 2—Mechanical Disruption to Telease Host Nucleic Acids

To assess whether mechanical disruption was an effective way to deplete the human nucleic acids present in the biological sample, 500 μl of a clinical sample taken from a subject was added to an MP biomatrix Lysis matrix tubes D, sourced from MP Biomedicals™. When the clinical sample underwent a preliminary slow centrifugation step, 500 μl of the resulting supernantant was added to an MP biomatrix Lysis matrix tubes D, sourced from MP Biomedicals™. The Lysis matrix tubes D are used for mechanical disruption of the host (e.g. human) cells present in the sample. The tube contains 1.4 mm ceramic, glass or stainless steel spheres or beads. Ceramic beads are preferred, however. The beads selectively disrupt mammalian cells while maintaining microbial cells intact. The average bead size is 1.2-1.6 mm, the bead capacity is 2 mL, and the Vickers Hardness is 800.

Lysing Matrix tubes D can be used lysis of softer tissues like the brain, liver, kidney, lung and spleen. The sample volume recommended is up to 200 mg (≤200 mg) of tissue (fresh; frozen; or dried, etc.), up to 200 μl (≤200 μl) of cells suspended in water or isotonic saline solution, up to 100 mg (≤100 mg) wet weight of tissue culture cells grown in suspension or up 107 (≤107) mammalian cells.

Lysis matrix tubes D are ideal for sample bead-beating using the TissueLyser LT, which provides fast, effective mechanical disruption for up to 12 samples concurrently. Simultaneous disruption and homogenization are achieved through high-speed shaking of samples in 2 ml microcentrifuge tubes with stainless steel, ceramic or glass beads. The TissueLyser LT can be combined with the coolable TissueLyser LT Adapter, which holds tubes during the disruption process.

The sample was bead-beaten for 3 minutes at 50 oscillations per second (OSC/sec) using a TissueLyser. Different times and speeds were tested for this step, and the optimal time/speed combination was determined to be 3 minutes at 50 OSC/sec.

Once the host cells had been mechanically disrupted, an endonuclease treatment was then performed to remove the human DNA from the sample.

### Step 3—Nuclease Activity

The method uses non-specific endonuclease enzymes to digest the host's nucleic acids. These enzymes are known to require high concentrations of salts, in particular, sodium chloride (NaCl), to function effectively. However, high NaCl concentrations may also negatively affect the microorganisms present in the sample, as NaCl can kill some of them because of its dehydrating effects on the microorganism cells. Therefore, to preserve the pathogen community present in the sample, the inventors have optimised the method such that the endonuclease activity requires little to no salt to function effectively.

The enzyme HL-SAN was selected, as it is a non-specific endonuclease active in various buffers and can be easily inactivated by treatment with a reducing agent. These features make HL-SAN particularly useful in protein purification and removal of both DNA and RNA from molecular biology reagents. Nucleic acids, especially genomic DNA, often pose a problem in the purification of DNA-binding proteins as they interfere with purification, downstream analysis or applications. In addition, contrary to most endonucleases, HL-SAN exhibits an optimum activity at high salt concentrations.

The high salt-tolerance and easy removal make HL-SAN beneficial to use in protein purification schemes, microorganism bioprocessing, PCR carry-over prevention, isothermal amplification, contamination control in RT-lamp, removal of genomic DNA from RNA preparations, decontamination of PCR master mix, PCR product clean-up, dephosphorylation before cloning, complete removal of DNA and RNA, and viscosity reduction. The HL-SAN enzyme used in this experiment was sourced from ArcticZymes technologies.

Given that the HL-SAN enzyme is known to reach the optimum nuclease activity (i.e. 100% activity) when the NaCl concentration is 0.5 M and a minimum enzyme activity (i.e. 10%) when this concentration is 0.5 M, the inventors assessed different concentrations of NaCl in the test mixture. The inventors determined which NaCl concentration (i.e. no salt, 0.5 M or 1 M) provides the optimum enzyme activity while maintaining an acceptable human DNA depletion, i.e. cycle threshold (ct) loss values superior to 7 (>7) or human DNA ct values superior to 30 (>30), and minimum DNA and RNA lost from the microorganisms present in the sample, i.e. ct loss values inferior to 2 (<2). A zeptometrix control which contains microorganism's RNA and DNA was used to assess RNA and DNA loss, whereas fifteen (15) BAL samples containing human DNA were used to determine human DNA depletion.

200 μl of the bead-beaten sample were transferred to a new conventional Eppendorf tube, and 10 μl of HL-SAN enzyme was added. The mixture was then heated for 10 minutes at 1000 rpm at 37° C. in an Eppendorf ThermoMixer shaker.

### Step 4—Micro-Organism's Nucleic Acid Extraction

Once the enzyme had digested the human nucleic acids, the mixture was transferred to the Roche Magnapure nucleic acid extraction instrument, and the microorganism nucleic acid extraction was performed as per the provider's standard protocol.

### Step 5—Detecting or Sequencing the Target Micro-Organism's Nucleic Acid

LunaScript RT SuperMix was added to the extracted sample to convert RNA into cDNA, followed by the double-strand synthesis of the DNA. The sample was then barcoded using the Rapid PCR barcoding kit (SQK-RPB004 Oxford Nanopore) and subsequently sequenced in a MinIOn flowcell (Oxford Nanopore) for 24 hours.

The above method was applied to different clinical samples, some of which were further spiked with pathogenic microorganisms.

### Results

The innovative detection method described herein is specifically designed to considerably deplete the host organism's genetic material, while ensuring minimal loss of the genetic material of the different pathogen(s) also present in the sample, and which therefore is to be detected. The method is non-selective for the infecting organism's nucleic acid and enables the detection of both DNA and RNA which may be simultaneously present in the sample.

### Example 1—Optimisation of the Enzymatic Activity Mediated by HL-SAN

As described above, the inventors aimed to preserve the pathogen community present in the biological sample taken from the subject, and therefore, optimised the method to enable the use of the minimum amount of salt possible.

In nucleic acid amplification tests, a positive reaction is detected by the accumulation of a fluorescent signal. The Ct (cycle threshold) is defined as the number of cycles required for the fluorescent signal to cross the threshold (i.e. exceeds background level). Ct levels are inversely proportional to the amount of target nucleic acid present in the sample, which means that the lower the Ct level, the greater the quantity of target nucleic acid present in the sample. The Ct loss values are proportional to the amount of target nucleic acids lost during the process (i.e. the lower the Ct loss value, the lower the loss of target nucleic acids; conversely, higher Ct loss values correspond to higher loss or depletion of the target nucleic acid).

Tables 1, 2 and 3 below show the method's accuracy relative to the enzyme activity at different concentrations of NaCl.

TABLE 1
HL-SAN activity without NaCl

| | | | Ct before | Ct after | |
|---|---|---|---|---|---|
| | Nucleic | NaCl | Host DNA | Host DNA | |
| Nucleic Acid source | acid type | concentration | Depletion | Depletion | Ct loss |
| Influenzavirus A | RNA | No buffer | 30.8 | 31.7 | 0.9 |
| Influenzavirus B | RNA | No buffer | 27.6 | 28.4 | 0.8 |
| Respiratory syncytial | RNA | No buffer | 31.4 | 32.5 | 1.1 |
| virus | | | | | |
| Parainfluenzavirus 1 | RNA | No buffer | 38.6 | 39.3 | 0.7 |
| Parainfluenzavirus 2 | RNA | No buffer | 34 | 34.9 | 0.9 |
| Parainfluenzavirus 3 | RNA | No buffer | 40.4 | ND* | N/A |
| Parainfluenzavirus 4 | RNA | No buffer | 34 | 34.3 | 0.3 |
| Adenovirus | DNA | No buffer | 25.3 | 28 | 2.7 |
| Metapneumovirus | RNA | No buffer | 28.9 | 29.9 | 1 |
| Rhinovirus | RNA | No buffer | 37.3 | 40.3 | 3 |
| Coronavirus 1 | RNA | No buffer | 33.1 | 33.8 | 0.7 |
| Coronavirus 2 | RNA | No buffer | 38.4 | 36.9 | −1.5 |
| Coronavirus 3 | RNA | No buffer | 33.1 | 34.3 | 1.2 |
| Chlamydia | DNA | No buffer | 35.5 | 35.6 | 0.1 |
| Mycoplasma | DNA | No buffer | 35.03 | ND* | N/A |
| BAL 16 | Human DNA | No buffer | 21 | 33 | 12 |
| BAL 17 | Human DNA | No buffer | 25 | 35 | 10 |
| BAL 18 | Human DNA | No buffer | 27 | 37 | 10 |
| BAL 19 | Human DNA | No buffer | 27 | 37 | 10 |
| BAL 20 | Human DNA | No buffer | ND | ND | N/A |

TABLE 2
HL-SAN activity with 0.5M of NaCl

| | | | Ct before | Ct after | |
|---|---|---|---|---|---|
| | Nucleic | NaCl | Host DNA | Host DNA | |
| Nucleic Acid source | acid type | concentration | Depletion | Depletion | Ct loss |
| Influenzavirus A | RNA | 0.5M | 26.5 | 29 | 2.5 |
| Influenzavirus B | RNA | 0.5M | 27 | 29 | 2 |
| Respiratory syncytial | RNA | 0.5M | 30 | 32.5 | 2.5 |
| virus | | | | | |
| Parainfluenza 1 | RNA | 0.5M | 34 | 36 | 2 |
| Parainfluenza 2 | RNA | 0.5M | 33 | 35 | 2 |
| Parainfluenza 3 | RNA | 0.5M | 35 | 38 | 3 |
| Parainfluenza 4 | RNA | 0.5M | 30 | 33 | 3 |
| Adenovirus | DNA | 0.5M | 26 | 30 | 4 |
| Metapneumovirus | RNA | 0.5M | 30 | 33 | 3 |
| Rhinovirus | RNA | 0.5M | 35 | 40 | 5 |
| Coronavirus 1 | RNA | 0.5M | 30 | 32 | 2 |
| Coronavirus 2 | RNA | 0.5M | 32 | 37 | 5 |
| Coronavirus 3 | RNA | 0.5M | 29 | 31.5 | 2.5 |
| Chlamydia | DNA | 0.5M | 31 | 34 | 3 |
| Mycoplasma | DNA | 0.5M | 31 | 34 | 3 |
| Influenza virus A | RNA | 0.5M | 26.5 | 28 | 1.5 |
| Respiratory syncytial | RNA | 0.5M | 29 | 30.6 | 1.6 |
| virus | | | | | |
| Parainfluenza 2 | RNA | 0.5M | 32 | 34 | 2 |
| Adenovirus | DNA | 0.5M | 24.5 | 28 | 3.5 |
| BAL 11 | Human DNA | 0.5M | 26 | ND* | N/A |
| BAL 12 | Human DNA | 0.5M | 20 | ND* | N/A |
| BAL 13 | Human DNA | 0.5M | 25 | ND* | N/A |
| BAL 14 | Human DNA | 0.5M | 22 | ND* | N/A |
| BAL 15 | Human DNA | 0.5M | ND | ND* | N/A |

TABLE 3
HL-SAN activity with 1M of NaCl

| | | | Ct before | Ct after | |
|---|---|---|---|---|---|
| | Nucleic | NaCl | Host DNA | Host DNA | |
| Nucleic Acid source | acid type | concentration | Depletion | Depletion | Ct loss |
| Influenzavirus A | RNA | 1M | 25.6 | 27.8 | 2.2 |
| Infleunzavirus B | RNA | 1M | 26.5 | 29.1 | 2.6 |
| Respiratory syncytial | RNA | 1M | 30.5 | 33.8 | 3.3 |
| virus | | | | | |
| Parainfluenzavirus 1 | RNA | 1M | 32.6 | 34.9 | 2.3 |
| Parainfleunzavirus 2 | RNA | 1M | 33.4 | 36.6 | 3.2 |
| Parainfleunzavirus 3 | RNA | 1M | 34 | 37 | 3 |
| Parainfleunzavirus 4 | RNA | 1M | 28.9 | 30.8 | 1.9 |
| Adenovirus | DNA | 1M | 24.6 | 27.3 | 2.7 |
| Metapneumovirus | RNA | 1M | 28.2 | 29.78 | 1.58 |
| Rhinovirus | RNA | 1M | 35.7 | 37.7 | 2 |
| Coronavirus 1 | RNA | 1M | 28.9 | 32 | 3.1 |
| Coronavirus 2 | RNA | 1M | 31.5 | 34.9 | 3.4 |
| Coronavirus 3 | RNA | 1M | 27.6 | 30.8 | 3.2 |
| Chlamydia | DNA | 1M | 30.2 | 32.1 | 1.9 |
| Mycoplasma | DNA | 1M | 29.8 | 32.5 | 2.7 |
| Influenzavirus A | RNA | 1M | 26.9 | 30 | 3.1 |
| BAL 6 | Human DNA | 1M | 26.3 | ND* | N/A |
| BAL 7 | Human DNA | 1M | 20 | ND* | N/A |
| BAL 8 | Human DNA | 1M | 34 | ND* | N/A |
| BAL 9 | Human DNA | 1M | 25 | ND* | N/A |
| BAL 10 | Human DNA | 1M | 35 | ND* | N/A |

As can be seen in the Tables, all of the microorganisms present in the positive controls were successfully detected in the amplification tests for all concentrations of NaCl tested. However, minimal microorganism Ct loss coupled with higher human DNA depletion was observed in tests where no NaCl was added (see Table 1). Therefore, the method has been successfully optimised to require no salt to while maintaining high levels of enzyme activity. As mentioned herein, high NaCl concentrations can often negatively affect the microorganisms present in the sample, as the salt can kill them due to its dehydrating effects. Therefore, to preserve the pathogenic population in the sample, for a better detection/sequencing, the inventors realised that it would be better to carry out the method at low (or zero) salt concentrations.

### Example 2—Host's DNA Depletion in Virus-Infected Samples

Once the method had been optimised so that it did not require the presence of (any) salt, the inventors investigated the method's effectiveness in depleting the host's DNA while maintaining substantial amounts of the nucleic acids of the infecting microorganisms present in the biological sample.

In a first experiment, negative BAL samples were spiked with a mixture of viral nucleic acids of different origins, including DNA and RNA. The samples were subjected to the method described above, and the results are summarised in Table 4 below.

TABLE 4
Human DNA depletion in BAL samples
spiked with positive viral control

| | | | Ct before | Ct after | |
|---|---|---|---|---|---|
| Sample | | Nucleic | Host DNA | Host DNA | Ct |
| number | Nucleic Acid source | acid type | Depletion | Depletion | lost |
| BAL 1 | Influenzavirus A | RNA | 26.5 | 28.3 | 1.8 |
| | Influenzavirus B | RNA | 28.5 | 29.7 | 1.2 |
| | Parainfluenzavirus 1 | RNA | 35.5 | 39.8 | 4.3 |
| | Parainfluenzavirus 2 | RNA | 34.1 | 35.1 | 1 |
| | Parainfluenzavirus 3 | RNA | 30.7 | 33.4 | 2.7 |
| | Parainfluenzavirus 4 | RNA | 32.2 | 34.6 | 2.4 |
| | Adenovirus | DNA | 27.8 | 32.9 | 5.1 |
| | Metapneumovirus | RNA | 30 | 32.4 | 2.4 |
| | Coronavirus 3 | RNA | 32.2 | 33.7 | 1.5 |
| | Coronavirus 4 | RNA | 27.7 | 30.8 | 3.1 |
| | Human DNA | DNA | 28 | 40 | 12 |
| BAL 2 | Influenzavirus A | RNA | 27.7 | 29.3 | 1.6 |
| | Influenzavirus B | RNA | 27.6 | 29.5 | 1.9 |
| | Parainfluenzavirus 1 | RNA | 34.6 | 38.1 | 3.5 |
| | Parainfluenzavirus 2 | RNA | 32.5 | 35.3 | 2.8 |
| | Parainfluenzavirus 3 | RNA | 30.7 | 32.3 | 1.6 |
| | Parainfluenzavirus 4 | RNA | 32.2 | 34.3 | 2.1 |
| | Adenovirus | DNA | 27.2 | 30.8 | 3.6 |
| | Metapneumovirus | RNA | 28.4 | 31.4 | 3 |
| | Coronavirus 4 | RNA | 27.5 | 34.1 | 6.6 |
| | Human DNA | DNA | 28 | 36 | 8 |
| BAL 3 | Influenzavirus A | RNA | 26.1 | 29.2 | 3.1 |
| | Influenzavirus B | RNA | 27.9 | 29.9 | 2 |
| | Parainfluenzavirus 2 | RNA | 33.1 | 36 | 2.9 |
| | Parainfluenzavirus 3 | RNA | 30.4 | 33.9 | 3.5 |
| | Parainfluenzavirus 4 | RNA | 32.3 | 36 | 3.7 |
| | Adenovirus | DNA | 27.7 | 33.4 | 5.7 |
| | Metapneumovirus | RNA | 29.2 | 32.3 | 3.1 |
| | Coronavirus 1 | RNA | 32 | 32 | 0 |
| | Coronavirus 4 | RNA | 27.6 | 31.5 | 3.9 |
| | human DNA | DNA | 24 | 37 | 13 |
| BAL 4 | Influenzavirus A | RNA | 28.7 | 30.5 | 1.8 |
| | Influenzavirus B | RNA | 28.6 | 30.2 | 1.6 |
| | Parainfluenzavirus 1 | RNA | 36.1 | 38.1 | 2 |
| | Parainfluenzavirus 2 | RNA | 33.3 | 34.5 | 1.2 |
| | Parainfluenzavirus 3 | RNA | 32.5 | 32.6 | 0.1 |
| | Parainfluenzavirus 4 | RNA | 32.4 | 33.4 | 1 |
| | Adenovirus | DNA | 27.9 | 29.5 | 1.6 |
| | Metapneumovirus | RNA | 29.3 | 30.9 | 1.6 |
| | Coronavirus 3 | RNA | 32.2 | 32.2 | 0 |
| | Human DNA | DNA | 20 | 37 | 17 |

As observed in Table 4 above, significant human (i.e. host) DNA depletion can be observed in each of the samples tested, whereas minimal loss of the viral nucleic acids occurred. Therefore, the inventors have confirmed that the method successfully depletes the host DNA in BAL samples, enabling the robust detection and characterization of the infecting viral nucleic acids (DNA and RNA) in the same samples.

In a subsequent experiment, the inventors tested the method on positive clinical nasal swabs. These samples were not spiked with pathogenic nucleic acids, but rather isolated from hosts naturally infected with different viruses. Table 5 below summarises the results observed with these clinical samples.

TABLE 5
Human DNA depletion in positive viral nasal swabs

| Nasal | | | | |
|---|---|---|---|---|
| Sample | Nucleic Acid | Ct before Host | Ct after Host | |
| Number | source | DNA Depletion | DNA Depletion | Ct lost |
| 1 | Sars-cov2 | 21/25 | 21/25 | 0 |
| | Human | 32 | 34 | 2 |
| | 16S | 24 | 24 | 0 |
| 2 | RSV | 24 | 26 | 2 |
| | Human | 23 | 28 | 5 |
| | 16S | 18 | 18 | 0 |
| 3 | RV_EV | 27 | 30 | 3 |
| | Human | 24 | 33 | 9 |
| | 16S | 10 | 10 | 0 |
| 4 | Sars-cov2 | 20.5/22.5 | 23/25 | 2.5/2.5 |
| | Human | 26 | 31 | 5 |
| | 16S | 14 | 15 | 1 |
| 5 | Sars-cov2 | 15/17 | 15/18 | 0/1 |
| | Human | 25 | 30 | 5 |
| | 16S | 12 | 14 | 2 |

Similarly to what was observed with the spiked BAL samples described above, the host's DNA was significantly depleted after the method was performed, whereas there was barely any loss in the target viral RNA nucleic acids which were also present in the clinical nasal swabs. The 16S data confirm that the bacterial DNA was not depleted even though the human DNA was depleted.

Therefore, the inventors have successfully demonstrated that the method of the invention is highly effective at depleting human DNA in clinical samples, but protecting the integrity of target RNA samples.

### Example 3—Host's DNA Depletion in Samples Infected with Mixed Pathogens To assess whether the method could successfully detect different pathogens simultaneously within the same sample, the inventors tested the method on positive clinical samples of various origins.

The samples were cultured to identify the microorganisms already present in the samples. Subsequently, the samples were spiked with additional microorganisms and the method of the invention was performed on each of the samples. The results observed under the above conditions are shown in FIG. 2A and 2B.

As can be seen in FIG. 2A, each of the isolates cultured was detected in the samples (see organism sequenced-number of sequence reads in FIG. 2). Furthermore, the method successfully detected both RNA and DNA of different pathogens, including viruses, bacteria and fungi.

In FIG. 2B, BAL samples were spiked with a mixture of organisms followed by PCR prior to depletion to establish each individual Ct value of the spiked organism. The depletion was carried out and then the sample was tested by PCR again to establish organism loss.

As with the other experiments described herein, the method successfully depleted human DNA in multi-pathogenic samples while conserving the nucleic acids of all the pathogenic microorganism communities that were also present in the samples (RNA and/or DNA).

### Example 4—Host's DNA Depletion in Lower Respiratory Samples

To assess the method's efficacy in depleting host's DNA in the samples, 21 lower respiratory samples were tested. The depletion was assessed by human-targeted PCR in sample aliquots before and after the depletion. Table 6 shows the CT values before and after the depletion.

TABLE 6
CT values in lower respiratory samples before and after
human DNA depletion. The following acronyms used in the
Table are defined as follows: PF = Pleural fluid;
BAL = Bronchi alveolar lavage, ETT = endothracheal aspirate.

| | | CT value for | CT value for | |
|---|---|---|---|---|
| Sample | Sample | the undepleted | the depleted | |
| number | type | aliquot | aliquot | ΔCT |
| 1 | PF | 22 | 30 | 8 |
| 2 | BAL | 22 | 33 | 11 |
| 3 | PF | 22 | 33 | 11 |
| 4 | PF | 22 | 34 | 12 |
| 5 | PF | 34 | 45 | 11 |
| 6 | PF | 20 | 32 | 12 |
| 7 | BAL | 24 | 45 | 21 |
| 8 | Sputum | 22 | 34 | 12 |
| 9 | BAL | 30 | 36 | 6 |
| 10 | PF | 22 | 26 | 4 |
| 11 | BAL | 22 | 45 | 23 |
| 12 | BAL | 24 | 33 | 9 |
| 13 | PF | 24 | 30 | 6 |
| 14 | PF | 25 | 38 | 13 |
| 15 | BAL | 33 | 45 | 12 |
| 16 | ETT | 32 | 45 | 13 |
| 17 | BAL | 27 | 37 | 10 |
| 18 | BAL | 28 | 45 | 17 |
| 19 | BAL | | 27 | 27 |
| 20 | BAL | 30 | 40 | 10 |
| 21 | Sputum | 28 | 40 | 12 |

### Example 5—Host's DNA Depletion in Upper Respiratory Samples

The method's efficacy in depleting host's DNA in the samples was also assessed in 28 upper respiratory samples. The depletion was assessed by human-targeted PCR in sample aliquots before and after the depletion. Table 7 shows the CT values before and after the depletion.

TABLE 7
CT values in upper respiratory samples
before and after human DNA depletion.

| | | CT value for | CT value for | |
|---|---|---|---|---|
| Sample | | the undepleted | the depleted | |
| number | Sample type | aliquot | aliquot | ΔCT |
| 22 | Nasal/Throat swab | 28 | 32 | 4 |
| 23 | Nasal/Throat swab | 28 | 32 | 4 |
| 24 | Nasal/Throat swab | 28 | 40 | 12 |
| 25 | Nasal/Throat swab | 28 | 38 | 10 |
| 26 | Nasal/Throat swab | 24 | 34 | 10 |
| 27 | Nasal/Throat swab | 26 | 34 | 8 |
| 28 | Nasal/Throat swab | 27 | 34 | 7 |
| 29 | Nasal/Throat swab | 26 | 35 | 9 |
| 30 | Nasal/Throat swab | 24 | 30 | 6 |
| 31 | Nasal/Throat swab | 26 | 37 | 11 |
| 32 | Nasal/Throat swab | 26 | 34 | 8 |
| 33 | Nasal/Throat swab | 26 | 33 | 7 |
| 34 | Nasal/Throat swab | 26 | 45 | 19 |
| 35 | Nasal/Throat swab | 29 | 35 | 6 |
| 36 | Nasal/Throat swab | 32 | 45 | 13 |
| 37 | Nasal/Throat swab | 28 | 36 | 8 |
| 38 | Nasal/Throat swab | 28 | 35 | 7 |
| 39 | Nasal/Throat swab | 31 | 35 | 4 |
| 40 | Nasal/Throat swab | 30 | 32 | 2 |
| 41 | Nasal/Throat swab | 28 | 33 | 5 |
| 42 | Nasal/Throat swab | 30 | 35 | 5 |
| 43 | Nasal/Throat swab | 30 | 37 | 7 |
| 44 | Nasal/Throat swab | 29 | 33 | 4 |
| 45 | Nasal/Throat swab | 28 | 45 | 17 |
| 46 | Nasal/Throat swab | 30 | 45 | 15 |
| 47 | Nasal/Throat swab | 29 | 45 | 16 |
| 48 | Nasal/Throat swab | 32 | 34 | 2 |
| 49 | Nasal/Throat swab | 29 | 34 | 5 |
| 50 | Nasal/Throat swab | 27 | 37 | 10 |

### Example 6—The Method's Limit of Detection (LOD).

To determine the method's limit of detection, BAL samples aliquoted in triplicates were spiked with different concentrations of K. pneumoniae (gram-negative bacterium), S. aureus (gram-positive bacteria), Influenza virus A (RNA viruse), Herpesvirus 6 (DNA virus) and C. albicans (yeast). Table 8 shows the detection limit of the method for each pathogen.

TABLE 8
Detection limit of the method in BAL samples spiked
with different concentrations of various pathogens.

| | | LoD | | LoD | |
|---|---|---|---|---|---|
| Microorganisms tested | | (low background) | | (high background) | |
| Bacteria | K. pneumoniae | 10\hat{ }3 | CFU/mL | 10\hat{ }3 | CFU/mL |
| | S. aureus | 10\hat{ }3 | CFU/mL | 10\hat{ }4 | CFU/mL |
| Virus | Influenza virus A | 70 | copies/mL | 70 | copies/mL |
| | HHV-6 | 10 | copies/mL | 10 | copies/mL |
| Fungi | C. albicans | 10\hat{ }3 | CFU/mL | 10\hat{ }3 | CFU/mL |

### Discussion and Conclusions

As demonstrated in the Examples, the inventors have developed a highly innovative and surprisingly effective metagenomic method that enables the rapid identification and characterisation of infections and can be applied to investigate the emergence of novel or rare pathogens. The innovative method detects both RNA and DNA of different microorganisms (i.e. viruses, bacteria, fungi and protozoans) in a single tube and can be implemented in routine diagnostic laboratories or hospitals near patients during outbreaks. The method complements the effort currently implemented in the bioinformatics field to facilitate rapid unbiased surveillance of new or unexpected pathogens close to the clinical front line where the first few cases are seen.

In addition, the detection limits of the metagenomic method of the invention is surprisingly good in either low and high backgrounds.

1. A method for detecting a microorganism in a biological sample obtained from a mammalian host, the sample comprising mammalian host cells and a microorganism, the method comprising:
(i) subjecting the biological sample to mechanical disruption such that the mammalian host cells are lysed to thereby release host's nucleic acid while leaving the microorganism substantially intact;
(ii) contacting the mechanically disrupted sample with a nuclease, to thereby digest the host's released nucleic acid while leaving the microorganism's nucleic acid substantially intact;
(iii) extracting the micro-organism's nucleic acid from the sample; and
(iv) detecting the extracted micro-organism's nucleic acid,
wherein the mechanical disruption step is achieved by contacting the sample with a plurality of particles, and then agitating the resultant sample for sufficient time and at sufficient intensity so that the particles cause the mammalian host cells to lyse, thereby releasing their nucleic acid.

2. A method according to claim 1, wherein the sample is a respiratory sample, optionally pleural fluid (PF), Bronchoalveolar lavage (BAL), sputum, non-direct Bronchoalveolar lavage (NDL), or a nose, mouth or throat swab, and/or wherein the host is a human being.

3. (canceled)

4. A method according to claim 1, wherein the method comprises initially centrifuging the biological sample before it is subjected to the mechanical disruption, optionally wherein the centrifugation step is conducted at between 300 g and 2000 g for at least 1 minute, or between 500 g and 1800 g for at least 5 minutes, or preferably between 1000g and 1400 g for at least 8 minutes.

5. A method according to claim 1, wherein at least 100, 500, 1000, 2000, 3000 or 5000 particles are used.

6. A method according to claim 1, wherein the particles comprise stainless steel, ceramic or glass.

7. A method according to clam 1, wherein the particles comprise ceramic.

8. A method according to claim 1, wherein: (i) the average diameter of the particles is between about 1 mm and 2 mm, or between about 1.1 mm and 1.8 mm, or between about 1.2 mm and 1.6 mm, or between about 1.3 mm and 1.5 mm; (ii) the average particle capacity is between 0.5 and 5 mL, or between 1 and 4 mL, or between 1.5 and 3 mL; and/or the average particle hardness is Vickers Hardness is 800.

9. (canceled)

10. (canceled)

11. A method according to claim 1, wherein the sample is agitated at:
(i) at least 5, 10, 15 or 20 oscillations per second (OSC/sec);
(ii) at least 25, 30 or 35 oscillations per second (OSC/sec);
(iii) at least 40, 45 or 50 oscillations per second (OSC/sec); and/or
(iv) less than 200, 150, 100 or 75 oscillations per second (OSC/sec).

12. A method according to claim 11, wherein the sample is agitated for:
(i) at least 15, 30, 45 or 60 seconds;
(ii) at least 1 min and 15 s, 1 min and 30 s, 1 min and 45 s, or 2 mins;
(iii) at least 2 min and 15 s, 2 min and 30 s, 2 min and 45 s, or 3 mins; and/or
(iv) less than 30 min, 20 min, 10 min or 5 min.

13. A method according to claim 1, wherein the mechanical disruption step is carried out at room temperature.

14. A method according to claim 1, wherein:
(i) the nuclease has both DNase and RNase activity;
(ii) the nuclease is an endonuclease or an exonuclease, preferably an endonuclease; and/or (iii) the nuclease comprises HL-SAN DNase or MolDNase, or an active variant thereof.

15. (canceled)

16. (canceled)

17. A method according to claim 1, wherein the sample is subjected to mixing after the nuclease has been added, wherein mixing is achieved by spinning the sample and nuclease at at least 100 rpm, preferably at least 500 rpm, and more preferably at least 1000 rpm.

18. A method according to claim 1, wherein the concentration of salt in the nuclease digestion step is less than 2 M, or less than 1 M, or less than 0.75 M, or less than 0.5 M, or less than 0.25 M, or less than 0.1 M salt, or preferably without salt.

19. (canceled)

20. A method according to claim 1, wherein HL-SAN is used with little or no salt in step (ii).

21. A method according to claim 1, wherein the micro-organism's nucleic acid is extracted in step (iii) from the digested sample using any automatic instrument or any manual extraction kit, and/or wherein the detection step (iv) is non-specific.

22. (canceled)

23. A method according to claim 1, wherein the method comprises: (i) a step of sequencing the pathogenic microorganism's nucleic acid, thereby detecting the microorganism; (ii) initially converting its DNA or its RNA to cDNA, creating double stranded DNA, fragmenting the dsDNA, and then PCR amplifying the fragments; and/or (iii) a PCR reaction which comprises subjecting the DNA to (a) at least 15 cycles, preferably at least 18 cycles, more preferably at least 20 cycles; or (b) at least 23 cycles, preferably at least 25 cycles, more preferably at least 27 cycles, and most preferably at least 30 cycles.

24. (canceled)

25. (canceled)

26. A method according to claim 1, wherein the microorganism is selected from a bacterium, virus, fungus and/or protozoan, optionally wherein (i) the bacterium is a Gram positive or a Gram negative bacterium, or wherein the bacterium is a mycobacterium or a bacterium without a cell wall, or (ii) the virus is a DNA virus or an RNA virus.

27. (canceled)

28. (canceled)

29. A method for depleting host nucleic acid in a biological sample obtained from a mammalian host, the sample comprising mammalian host cells and a microorganism, the method comprising:
(i) subjecting the biological sample to mechanical disruption such that the mammalian host cells are lysed to thereby release host's nucleic acid while leaving the microorganism substantially intact; and
(ii) contacting the mechanically disrupted sample with a nuclease, to thereby digest and deplete the host's released nucleic acid while leaving the microorganism's nucleic acid substantially intact,
wherein the method is as defined in claim 1.

30. (canceled)

31. An apparatus for detecting a microorganism in a biological sample comprising mammalian host cells and a microorganism, the apparatus comprising:
a mechanical cell disruptor configured, in use, to mechanically disrupt a biological sample such that the mammalian host cells are lysed to thereby release host's nucleic acid while leaving the microorganism substantially intact; and
a nuclease configured, in use, to thereby digest the host's released nucleic acid while leaving the microorganism's nucleic acid substantially intact,
wherein the mechanical cell disruptor comprises a plurality of particles and a device for mechanically agitating the particles in the sample.

32. The apparatus according to claim 31, wherein the apparatus comprises reagents for detecting and optionally sequencing the microorganism's nucleic acid, and/or wherein the apparatus is configured to carry out a method for detecting the microorganism in the biological sample, wherein the method comprises:
(i) subjecting the biological sample to mechanical disruption such that the mammalian host cells are lysed to thereby release host's nucleic acid while leaving the microorganism substantially intact;
(ii) contacting the mechanically disrupted sample with a nuclease, to thereby digest the host's released nucleic acid while leaving the microorganism's nucleic acid substantially intact;
(iii) extracting the micro-organism's nucleic acid from the sample; and
(iv) detecting the extracted micro-organism's nucleic acid,
wherein the mechanical disruption step is achieved by contacting the sample with a plurality of particles, and then agitating the resultant sample for sufficient time and at sufficient intensity so that the particles cause the mammalian host cells to lyse, thereby releasing their nucleic acid.

33. (canceled)

34. (canceled)


SEQUENCE LISTING (13 sequences)

SEQ ID NO: 1 - DNA, 24 bp, synthetic construct
  source 1..24: mol_type = other DNA
aagaaagttgtcggtgtctttgtg

SEQ ID NO: 2 - DNA, 24 bp, synthetic construct
  source 1..24: mol_type = other DNA
tcgattccgtttgtagtcgtctgt

SEQ ID NO: 3 - DNA, 24 bp, synthetic construct
  source 1..24: mol_type = other DNA
gagtcttgtgtcccagttaccagg

SEQ ID NO: 4 - DNA, 24 bp, synthetic construct
  source 1..24: mol_type = other DNA
ttcggattctatcgtgtttcccta

SEQ ID NO: 5 - DNA, 24 bp, synthetic construct
  source 1..24: mol_type = other DNA
cttgtccagggtttgtgtaacctt

SEQ ID NO: 6 - DNA, 24 bp, synthetic construct
  source 1..24: mol_type = other DNA
ttctcgcaaaggcagaaagtagtc

SEQ ID NO: 7 - DNA, 24 bp, synthetic construct
  source 1..24: mol_type = other DNA
gtgttaccgtgggaatgaatcctt

SEQ ID NO: 8 - DNA, 24 bp, synthetic construct
  source 1..24: mol_type = other DNA
ttcagggaacaaaccaagttacgt

SEQ ID NO: 9 - DNA, 24 bp, synthetic construct
  source 1..24: mol_type = other DNA
aactaggcacagcgagtcttggtt

SEQ ID NO: 10 - DNA, 24 bp, synthetic construct
  source 1..24: mol_type = other DNA
aagcgttgaaacctttgtcctctc

SEQ ID NO: 11 - DNA, 24 bp, synthetic construct
  source 1..24: mol_type = other DNA
gtttcatctatcggagggaatgga

SEQ ID NO: 12 - DNA, 24 bp, synthetic construct
  source 1..24: mol_type = other DNA
caggtagaaagaagcagaatcgga

SEQ ID NO: 13 - DNA, 24 bp, synthetic construct
  source 1..24: mol_type = other DNA
gttgagttacaaagcaccgatcag